(19) United States
(12) Patent Application Publication
(10) Pub. No.: US 2025/0130121 A1
(43) Pub. Date: Apr. 24, 2025

(54) OPTICAL FIBER INSTRUMENTED APPARATUS AND METHODS OF USE THEREOF

(71) Applicant: The Texas A&M University System, College Station, TX (US)

(72) Inventors: Jonathan Tyler Gates, College Station, TX (US); Pavel Tsvetkov, College Station, TX (US)

(21) Appl. No.: 18/491,944

(22) Filed: Oct. 23, 2023

(51) Int. Cl. G01K 11/3206; G01K 7/02

(52) U.S. Cl. CPC G01K 11/3206; G01K 7/021; G01K 2213/00

(57) ABSTRACT

An instrumented blanket includes an insulative material and a temperature control structure integrated with the insulative material. The heating structure is configured to produce a heat or a cooling output in response to an input signal. The temperature-control blanket further includes an optical sensor including an optical fiber integrated with the insulative material along the heating structure. The optical sensor is configured to detect a change in an optical property measured from a plurality of intervals along a length of the optical fiber. The change in the optical property is indicative of a temperature of the temperature-control blanket at a respective interval of the plurality of intervals.

### TECHNICAL FIELD

The described examples relate generally to systems, devices, and techniques for measure and controlling parameters of an insulative blanket using optical fibers.

### BACKGROUND

Industrial processes, including those associated with energy production, often require fine control of fluid temperature and/or other parameters associated with process equipment and piping. For example, such processes may include inducing a flow of certain fluids that must be maintained above a certain temperature threshold. In conventional systems, an insulative blanket may be used to encompass a portion of the process equipment or piping in order to mitigate heat loss from the fluid held therein. In some cases, a heat trace and/or other element may be integrated with the insulative material in order to actively provide heat to the fluid. While such conventional systems may be used to maintain a temperature of the fluid, conventional blankets often fail to include any feedback mechanism that can determine the current temperature and/or other parameters of the blanket. Further, even where traditional thermocouples are used with such insulative blankets, thermocouples often fail to be useful for providing a high-resolution, 3-D images of the temperature of the process equipment/piping and associated fluids therein.

Certain industrial processes, such as those associated with molten salt nuclear reactors, may require fluid (e.g., fissile molten salt) to remain above a certain temperature across an entire 3-D volume of the process equipment or pipe within which the fluid is contained, else plugging or other failure mechanisms may result. Conventional insulative blanket systems may therefore fail to be capable of determining the 3-D temperature distribution of such processes, and in many cases, the operation of such conventional systems may be hindered or prevented due to the excess electromagnetic and/or radiological interference associated with nuclear reactors. As such, there remains a need for improved insulative blankets capable of temperature control and generating high-resolution temperature mapping of systems in harsh operating environments, such as those with the potential for high levels of electromagnetic and/or radiological interference.

### SUMMARY

In one example, an instrumented temperature-control blanket is disclosed. The blanket includes an insulative material. The blanket further includes a heating structure or other temperature control structure integrated with the insulative material that is configured to produce a heat and/or cooling output in response to an input signal. The blanket further includes an optical sensor including an optical fiber integrated with the insulative material along the heating structure. The optical sensor is configured to detect a change in an optical property measured from a plurality of intervals along a length of the optical fiber. The change in the optical property is indicative of a temperature of the temperature-control blanket at a respective interval of the plurality of intervals.

In another example, the optical sensor may further include a signal generator configured to propagate an optical signal through the optical fiber. The optical sensor may further include a receiver configured to detect returned light scattering, based on the optical signal, from each interval of the plurality of intervals of the optical fiber. Further, the optical sensor may be coupled with a processing unit. The processing unit may be configured to analyze the returned light scattering to determine the change in the optical property from each interval of the plurality of intervals along the length of the optical fiber. The processing unit may be further configured to determine the temperature of the temperature-control blanket for each interval of the plurality of intervals by comparing the change in the optical property for each respective interval with a baseline optical property.

In another example, the processing unit may be configured to analyze the returned light scattering using one or both of an optical time domain reflectometry or an optical frequency domain reflectometry.

In another example, the optical property may include a light frequency spectrum. In this regard, the processing unit may associate changes of the light frequency spectrum with each respective interval of the plurality of intervals based on a detected time of the returned light scattering at the receiver.

In another example, the insulative material may be wrapped around a pipe or a vessel. In this regard, the processing unit may be configured to generate a 3-D temperature map corresponding to a 3-D surface of the pipe or vessel engaged with the insulative material. The 3-D temperature map may be formed from the determined temperature of the temperature-control blanket at each respective interval.

In another example, the processing unit may be configured to compare the determined temperature of the temperature-control blanket to one or more set points. Further, the blanket may include a controller operatively coupled with the heating structure or other temperature control structure and configured to deliver the input signal thereto (e.g., an electrical signal, a flow of coolant or other medium, and so on). The controller may, in turn, be configured to change a property of the input signal based on the determined temperature of the temperature-control blanket deviating from the one or more set points.

In another example, the temperature control structure may include the heating structure, and the blanket may further include a first thermocouple assembly integrated with the insulative material along a first interval of the plurality of intervals of the optical fiber. The first thermocouple may be configured to measure a first temperature of the temperature-control blanket at the first interval. The blanket may further include a second thermocouple assembly integrated with the insulative material along a interval of the plurality of intervals of the optical fiber. The second thermocouple may be configured to measure a second temperature of the temperature-control blanket at the second interval. In some cases, the processing unit may be further configured to determine the baseline optical property by correlating the first measured temperature of the first thermocouple assembly and the second measured temperature of the second thermocouple assembly with the optical property measured by the optical sensor for said first and second temperatures.

In another example, the optical sensor may be a first optical sensor. In this regard, the blanket may further include a second optical sensor including a second optical fiber integrated with the insulative material along the heating structure and the first optical sensor. Further, the second optical sensor may be configured to detect a change in an optical property measured from a plurality of intervals along a length of the second optical fiber. The change in the optical property may be indicative of a temperature of the temperature-control blanket at a respective interval of the plurality of intervals of the second optical fiber.

In another example, the insulative material may include a high-temperature ceramic with formed channels therein configured to receive at least a portion of the heating structure and at least a portion of the optical fiber.

In another example, the blanket may further include high-temperature ceramic threads engaged with the insulative material and the portion of the heating structure and/or the portion of the optical fiber to secure the portion of the heating structure and/or the portion of the optical fiber in the formed channels.

In another example, the portion of the heating structure and the portion of the optical fiber may be enclosed within the insulative material.

In another example, the formed channels may define a serpentine pattern.

In another example, the heating structure may form a part of a temperature control structure integrated with the insulative material. The temperature control structure may further include a cooling structure integrated with the insulative material and configured to produce a cooling output in response to an input cooling signal. The cooling structure may include a cooling medium induced along or through a portion of the insulative blanket.

In another example, the processing unit may be configured to compare the determined temperature of the temperature-control blanket to one or more set points. In turn, the blanket may further include a controller operatively coupled with the temperature control structure that is configured to deliver the input electrical signal to the heating structure and the input cooling signal to the cooling structure. The controller may be further configured to change a property of the one or both of the input electrical signal and/or the input cooling signal based on the determined temperature of the temperature-control blanket deviating from the one or more set points.

In another example, a system is disclosed. The system includes a temperature control blanket, such as any of the temperature control blankets disclosed herein. The system further includes a pipe or a vessel having a fluid therein. The blanket may be engaged with the pipe or the vessel to provide the heat output of the heating structure to the fluid.

In another example, the blanket of said system may further include a signal generator configured to propagate an optical signal through the optical fiber. Further, the blanket of said system may include a receiver configured to detect returned light scattering, based on the optical signal, from each interval of the plurality of intervals of the optical fiber. The system may further include a processing unit configured to analyze the returned light scattering to determine the change in the optical property from each interval of the plurality of intervals along the length of the optical fiber. The processing unit of said system may be further configured to determine the temperature of the temperature-control blanket for each interval of the plurality of intervals by comparing the change in the optical property for each respective interval with a baseline optical property.

In another example, the fluid of said system may include a fissile molten salt fluid of a molten salt reactor system. In this regard, the processing unit of said system may be further configured to compare the determined temperature of the temperature control blanket to one or more set points associated with a freezing temperature of the fissile molten salt fluid. Further, the blanket of said system may further include a controller operatively coupled with the heating structure and configured to deliver the input electrical signal thereto. The controller may be configured to change a property of the input electrical signal based on the determined temperature of the temperature-control blanket advancing toward the one or more set points.

In another example, a method of operating an instrumented temperature-control blanket is disclosed. The method includes producing a temperature output from a temperature control structure in response to an input signal. The temperature control structure is integrated with an insulative material. The method further includes detecting, using an optical sensor including an optical fiber integrated with the insulative material along the temperature control structure, a change in an optical property measured from a plurality of intervals along a length of the optical fiber. The change in the optical property is indicative of a temperature of the temperature-control blanket at a respective interval of the plurality of intervals.

In another example, the method may further include propagating, using a signal generator of the optical sensor, an optical signal through the optical fiber. The method may further include detecting, using a receiver of the optical sensor, a returned light scattering, based on the optical signal, from each interval of the plurality of intervals of the optical fiber. The method may further include analyzing, using a processing unit coupled with the optical sensor, the returned light scattering to determine the change in the optical property from each interval of the plurality of intervals along the length of the optical fiber. The method may further include determining, using the processing unit, the temperature of the temperature-control blanket for each interval of the plurality of intervals by comparing the change in the optical property for each respective interval with a baseline optical property.

In another example, the method may further include comparing, using the processing unit, the determined temperature of the temperature control-blanket to one or more set points. The method may further include changing, using a controller operatively coupled to the temperature control structure and the processing unit, a property of input signal based on the determined temperature of the temperature-control blanket deviating from the one or more set points.

In addition to the example aspects described above, further aspects and examples will become apparent by reference to the drawings and by study of the following description.

### BRIEF DESCRIPTION OF THE DRAWINGS

FIG. 1 depicts a system including an instrumented temperature-control blanket and a pipe.

FIG. 2 depicts a 3-D temperature map indicative of a surface temperature of the pipe of FIG. 1.

FIG. 3 depicts a functional diagram of an example instrumented blanket.

FIG. 4 depicts a functional diagram of another example instrumented blanket.

FIG. 5 depicts a functional diagram of another example instrumented blanket.

FIG. 6 depicts a functional diagram of another example instrumented blanket.

FIG. 7 depicts a functional diagram of another example instrumented blanket.

FIG. 8 depicts a functional diagram of an example system including the instrumented blanket of FIG. 7.

FIG. 9 depicts a functional diagram of certain P&ID logic of the system of FIG. 8.

FIG. 10 depicts another example instrumented blanket.

FIG. 11 depicts an example insulative material of the instrumented blanket of FIG. 10.

FIG. 12A depicts an example heating structure of the instrumented blanket of FIG. 10.

FIG. 12B depicts an example, optional, cooling structure of the instrumented blanket of FIG. 10.

FIG. 13 depicts an example optical sensor of the instrumented blanket of FIG. 10.

FIG. 14 depicts an example optical fiber of the optical sensor of FIG. 13.

FIG. 15 depicts an example thermocouple of the instrumented blanket of FIG. 10.

FIG. 16 depicts a functional diagram of an example molten salt reactor system.

FIG. 17 depicts another example system including an instrumented blanket of the present disclosure and a battery.

FIG. 18 depicts a flow diagram of an example method of operating an instrumented blanket.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

The description that follows includes sample systems, methods, and apparatuses that embody various elements of the present disclosure. However, it should be understood that the described disclosure may be practiced in a variety of forms in addition to those described herein.

The following disclosure relates generally to an instrumented blanket, such as a “temperature-control” and/or parameter measuring blanket (e.g., including stress, strain, and/or neutron and gamma fluence). Broadly, a “blanket” as described herein may include any of a variety of insulative materials (e.g., ceramics, wool, and so forth) that are used to mitigate heat loss from fluids of certain industrial processes. For example, many industrial processes, including those associated with energy production, often require that fluid contained therein be maintained at or above certain temperatures (e.g., to maintain the fluid at a certain state, viscosity, and/or other parameter that is supportive of said industrial processes). Insulative blankets may be wrapped around or otherwise generally engaged with process equipment, piping, and/or other components of the processes to trap heat within process by providing a thermal barrier between the process and an external environment.

In some conventional cases, a heat trace and/or other element may be integrated with the insulative material of the blanket in order to actively provide heat to the process and associated fluids therein. Further, while many conventional blankets lack any feedback mechanism that can determine the current temperature and/or other parameters of the blanket, thermocouples have traditionally been used to provide a point-source measurement of blanket heat. Such conventional blanket systems fail to provide a high-resolution feedback mechanism that can be used to determine a 3-D heating mapping of the blanket and associated process equipment. Further, such conventional blanket systems fail to provide robust solutions for particularly harsh environments, such as those associated with nuclear reactor operations, which may have substantially elevated levels of electromagnetic and/or radiological interference that hinders the operation of conventional sensors. Accordingly, conventional blanket systems may be inoperative or unfunctional in certain harsh environments where a high-resolution view of a temperature of the blanket and associated process is required, such as those of associated with molten salt reactors. For example, and as described in greater detail herein, the molten salt reactor may require fluid (e.g., a fissile molten salt) to remain above a certain temperature across an entire 3-D volume of the process equipment or pipe within which the fluid is contained, else plugging or other failure mechanisms may result. Conventional blanket systems may therefore lack the ability to provide a high-resolution, 3-D mapping of the processing equipment or piping of such molten salt reactor systems, and therefore may not be capable of identifying hot or cold spots in the system that could indicate potential system failures.

To mitigate these and other challenges, the instrumented blanket of the present disclosure includes both a temperature control structure (e.g., a cooling and/or a heating structure) and an optical sensor integrated with the insulative material of the blanket. Broadly, the temperature control structure may include any appropriate collection of components, assemblies, and subassemblies configured to change a temperature of the blanket, and thus cause a corresponding change of temperature to any associated processing equipment associated with the blanket. In one example, and as described in greater detail herein, the temperature control structure may include a heating structure and/or other appropriate component configured to generate a heat output in response to a signal, such as an electrical signal. Sample heating structures include nickel/nichrome wire and/or other heat trace material that is operative to produce a heat output in response to a signal, such as an electric signal. Additionally or alternatively, the temperature control structure may include a cooling structure and/or other appropriate component configured to generate a cooling output in response to a signal, such as an electrical signal. Sampling cooling structures includes a flexible bladder or flexible conduit capable of routing a flow of a cooling medium therethrough in response to a receipt of a cooled flow of said medium into the flexible material. Accordingly, the temperature control structure may be used to maintain a temperature of associated process equipment within a desired range by either impart a heat output or a cooling output to said equipment.

The blanket of the present disclosure may further include the optical sensor. The optical sensor, as described in greater detail herein, may be used to provide a high-resolution 3-D temperature mapping of the blanket that is indicative of a temperature of processing equipment associated with the blanket. The optical sensor may be used to perform such functions in environments having substantially high electromagnetic and/or radiological interference, such as may be present in molten salt nuclear reactor systems. To facilitate the foregoing, the optical sensor may include an optical fiber integrated with the insulative material along a path of the heating structure. The optical sensor may, in turn, be configured to detect a change in an optical property measured from a plurality of intervals along a length of the optical fiber. The change in the optical property from each of the intervals may be indicative of a temperature of the insulative material and blanket at such respective interval. The plurality of temperature changes as measured across each of the intervals of the optical fiber may be processed to generated a 3-D mapping of a temperature of the blanket.

While many methods of operation of the optical sensor are possible and contemplated herein, in one example, the optical sensor includes a signal generator and a receiver. The signal generator may be configured to propagate an optical signal through the optical fiber. In turn, the receiver is configured to detect a returned light scatting, based on the optical signal, from each interval of the plurality of intervals of the optical fiber. The optical sensor may be operatively coupled with a processing unit or other computing device to determine the change of optical property and associated temperature change based on the return light scattering measured at the receiver. For example, the processing unit may analyze the returned light scattering to determine a change in the optical property from each interval of the plurality of intervals of the fiber using one or both of an optical time domain reflectometry or optical frequency domain reflectometry. The optical property may be a light frequency spectrum of the scattered light, the magnitude of which may be determined by said reflectometry methods. The processing unit may further associate the change in the light frequency spectrum with a time of receipt of the return light scattering in order to calculate a location of the fiber associated with the change in the light frequency spectrum. The processing unit may further determine the temperature of said location by comparing the change in the optical property for the respective interval with a baseline optical property (e.g., a known light frequency spectrum for a given temperature). Furthermore, in some cases, the processing unit may additionally or alternatively determine a strain, stress, and/or neutron or gamma fluence associated with said location by comparing of the foregoing parameters with a baseline optical property (e.g., a known light frequency spectrum for a given strain, stress, or neutron and gamma fluence).

The optical sensor may determine the temperature (or other parameters, such as stress, strain, or neutron or gamma fluence) of the blanket for each interval of the optical fiber. At least a portion of the optical fiber may be integrated with the insulative material, which is wrapped or otherwise engaged with the process equipment or piping of the associated process. In some cases, the portion of the optical fiber may be integrated with the insulative material in a serpentine pattern or other pattern in order to integrate the optical fiber across a wider surface area of the blanket. The geometry of the pipe or other process equipment around which the insulative material may be known. As such, the determined temperature (and/or other determined parameter) for each of said intervals of the optical fiber may be mapped onto the known geometry of the equipment in order to produce a generally high-resolution, 3-D mapping of the temperature of the equipment. In many cases, the 3-D temperature map may, in turn, be used to provide information that can be used to identify localized hot or cold spots in the process equipment (and/or other identify spots of interest associated with the determined parameters), which could indicate a failure mechanism or otherwise information associated with the associated process. For example, where used with a molten salt nuclear reactor system, the 3-D temperature map could be used to identify a localized cold spot at which the fissile molten salt has dropped below a freezing temperature, which could indicate a failure of the piping at said localized cold spot.

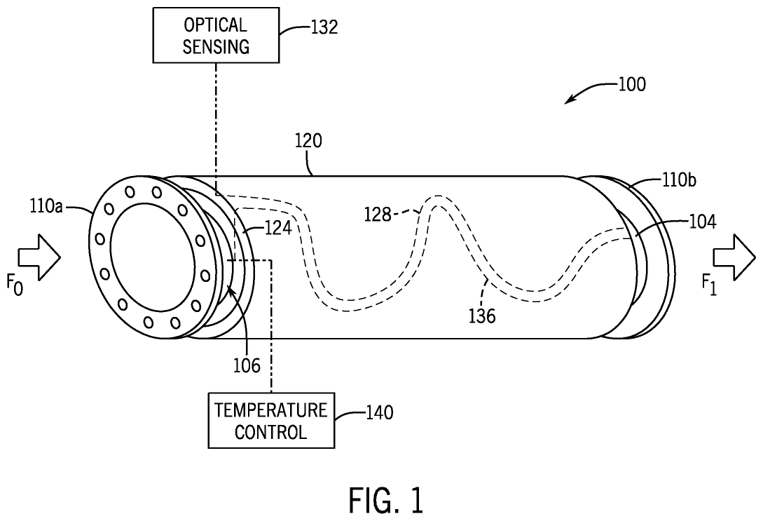

Turning to the Drawings, FIG. 1 depicts a system 100 including an instrumented blanket 120, such as the instrumented blankets discussed generally above and described in greater detail below. The system 100 is further shown as including a pipe 104 having a surface 106 and a opposing flanges 110a, 110b. The pipe 104 may be a component of an industrial process, such as an energy-producing process of a molten salt nuclear reactor, and include an initial fluid flow F₀ into the pipe 104 and an output fluid flow F₁ output form the pipe 104.

The blanket 120 may be used to maintain a temperature of the surface 106 of the pipe 104, and associated fluids contained therein. For example, the temperature-controlled blanket 120 may broadly include an insulative material 124 having an optical fiber 128 and a temperature control structure 136 integrated therewith. As described herein, the temperature control structure 136 may include any appropriate combination of heat output and/or cooling output producing components that may operate to cause a temperature change of the blanket 120 and associated surface 106 of the pipe 104. In this regard, the temperature control structure 136 may be operatively associated with a temperature control module 140 that may include a controller or other mechanism to deliver an input electrical signal or other input to the temperature control structure 136 to cause such temperature changes. Alternatively, it will be appreciated that the temperature control structure 136 and temperature control module 140 may be omitted; for example, such as where the instrument blanket 100 is used for sensing and measuring one or more parameters, including temperature, stress, strain, and/or neutron and gamma fluence.

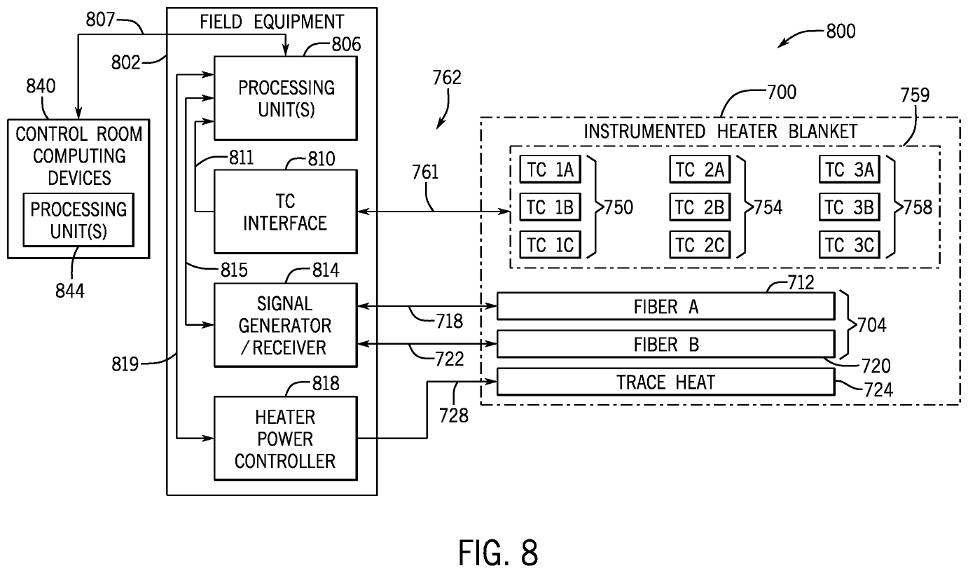

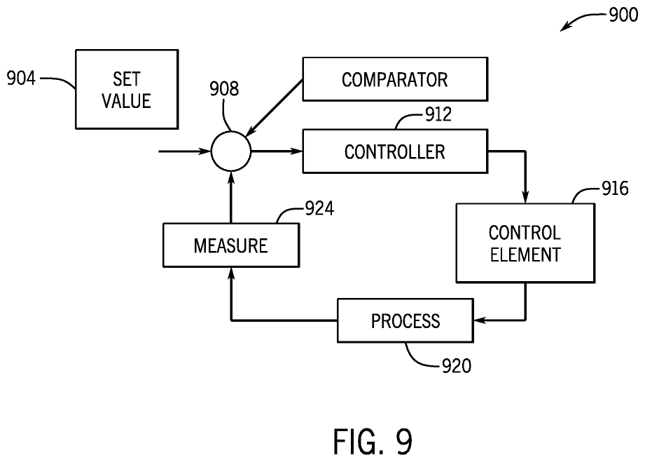

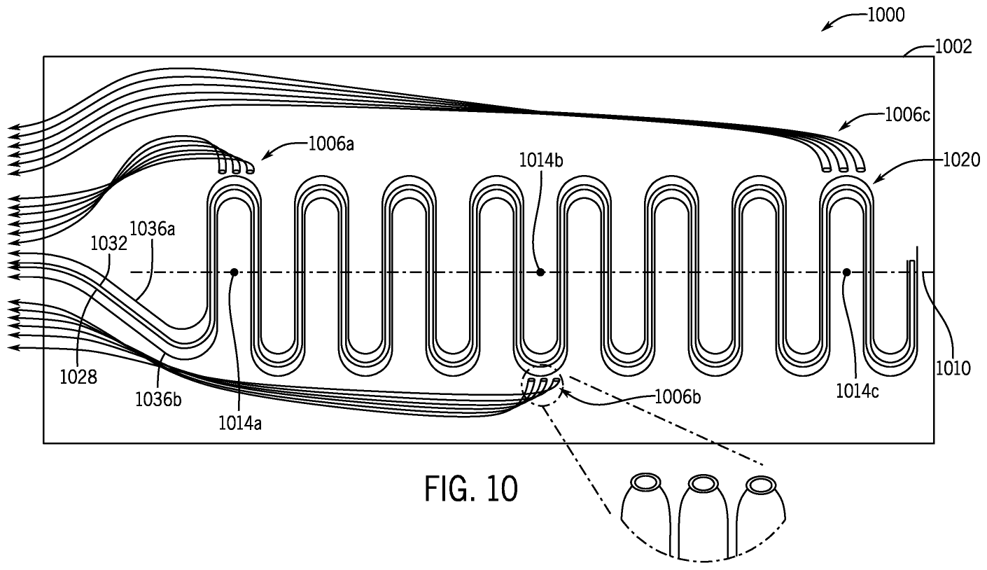

The optical fiber 128 may be a component of an optical sensor, such as any of the optical sensors described herein, that can detect changes of optical properties indicative of temperature changes and/or other parameters, such as those associated with stress, strain and/or neutron or gamma fluence. In this regard, the optical fiber 128 may be operatively associated with an optical sensing module 132 that is configured to deliver optical signals to the optical fiber, and to receive returned light scattering from said optical signals, which may be analyzed to determine parameter changes indicated by the light scattering. It will be appreciated that the temperature control module 140 and/or the optical sensing module 132 may be operatively coupled with a processing unit and/or other computing device (e.g., as shown and described herein in relation to FIGS. 8 and 9) in order to perform one or more functions described herein, including analyzing the light scattering of the optical sensing module 132 and determining said parameter (e.g., temperature changes), and causing one or more operations of the temperature control module 140 (where implemented) based on such determinations.

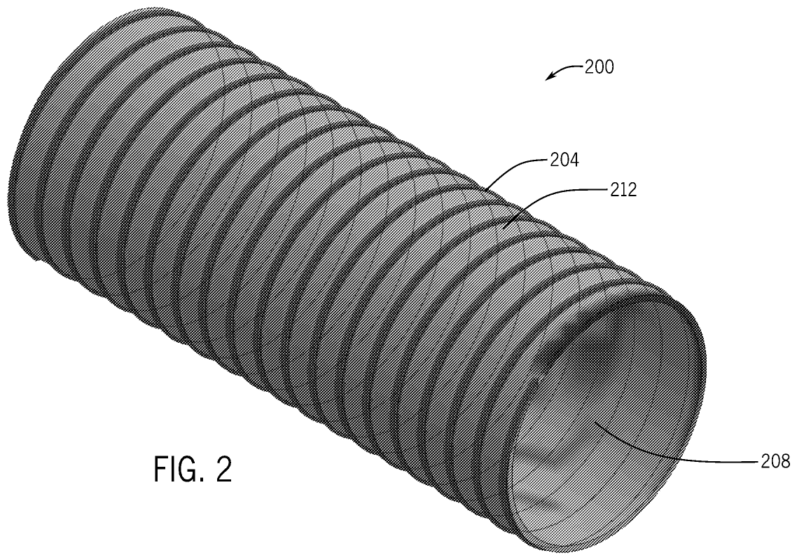

The operation of the instrumented blanket 120 may produce temperature data for each interval of a plurality of intervals along a length of the optical fiber 128. This temperature data may be mapped to a 3-D shape of the pipe 104 (or other appropriate structure) in order to provide a 3-D visualization of heat and temperature distribution across the surface 106. For example, and as shown in FIG. 2, a temperature map 200 of the pipe 104 may be provided using the temperature control-blanket 120. The temperature map 200 may indicate certain elevated temperature regions 204, reduced temperature regions 208, and target temperature regions 212, as shown in FIG. 2. In one example, the pipe 104 may be a pipe engaged to carry a fissile molten salt through a loop of a molten salt nuclear reactor system. Accordingly, the temperature map 200 may be used to identify portions of the pipe 104 at which the fissile molten salt may be approaching or have fallen below a freezing temperature for the salt, such as may be indicated by the reduced temperature regions 212. The blanket 120 may therefore be used to provide a more complete view of the heat and temperature distribution across the pipe 104, which may be used detect potential failures that are indicated via reduced or elevated temperatures (e.g., such as where the reduced temperature region 212 indicate potential plugging or leaks of the fissile molten salt from the pipe 104). Additionally or alternatively, analogous maps may be produced corresponding to one or more other measured parameters of the associated process, including maps that depict measured stress, strain, and/or neutron or gamma fluence measurements/determinations.

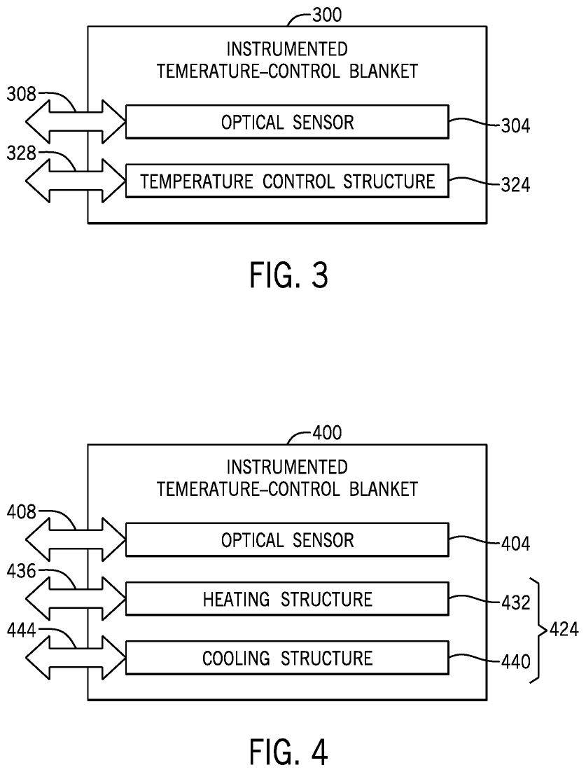

With reference to FIGS. 3-7, various functional diagrams of instrumented blankets of the present disclosure are depicted. While FIGS. 3-7 show examples of the various components and configuration of such blankets, it will be appreciated that more or different configurations are possible in which an optical sensor is used to detected temperature changes in an insulative blanket.

With reference to FIG. 3, an instrumented blanket 300 is depicted. The instrumented blanket 300 may include an optical sensor 304 and a temperature control structure 324. The optical sensor 304 may include any appropriate collection of components, assemblies, and subassemblies to support the determination of a temperature by on a change in an optical property. For example, the optical sensor 304 may include an optical fiber integrated with an insulative material of the blanket 300. The optical sensor 304 may propagate optical signals through the optical fiber and detect a returned light scattering from said light signals at each of a plurality of intervals along a length of the optical fiber. The returned light scattering may be analyzed by the optical sensor 304 (or a processing unit associated therewith via signal coupling 308) to determine changes in optical properties (such as changed in light frequency spectrums) associated with the light scattering. In some cases, an optical time domain reflectometry or an optical frequency domain reflectometry may be used to perform said analysis. The optical sensor 304 (or a processing unit associated therewith) may then determine a temperature of the blanket 300 for each interval of the plurality of intervals by comparing the change in the optical property to a baseline or temperature-calibrated optical property. Additionally or alternatively, in some cases, the optical sensor 304 may be used to measure or determine a strain, stress, and/or neutron or gamma fluence associated with each interval or location by comparing of the foregoing parameters with a baseline optical property (e.g., a known light frequency spectrum for a given strain, stress, or neutron or gamma fluence).

The temperature control structure 324 may include any appropriate collection of components, assemblies, and subassemblies configured to change a temperature of the blanket 300, and thus cause a corresponding change of temperature to any associated processing equipment associated with the blanket 300. For example, the temperature control structure 324 may include a heating structure and/or other appropriate component configured to generate a heat output in response to a signal, such as an electrical signal (which may be received via signal coupling 328). Additionally or alternatively, the temperature control structure 324 may include a cooling structure and/or other appropriate component configured to generate a cooling output in response to a signal, such as an electrical signal (which may be received via signal coupling 328). Accordingly, the temperature control structure 324 may be used to maintain a temperature of associated process equipment within a desire range by either impart a heat output or a cooling output to said equipment.

With reference to FIG. 4, an instrumented blanket 400 is shown. The instrumented temperature control blanket 400 may be substantially analogous to the instrumented blanket 300 of FIG. 3 and include an optical sensor 404, a signal coupling 428, and a temperature control structure 424. Notwithstanding the foregoing similarities, the instrumented blanket 400 is shown as including both a heating structure 432 (and associated signal coupling 436) and a cooling structure 440 (and associated signal coupling 444). In this regard, the instrumented blanket 400 is capable of producing both a heat output (from the heating structure 432) and a cooling output (from the cooling structure 440). The instrumented blanket 400 may therefore be capable of maintaining the insulative material of the blanket 400 and associated processing equipment within a set range; providing cooling via the cooling structure 440 when the optical sensor 404 measures temperature above certain set points, and providing heating vie the heating structure 432 when the optical sensor 404 measures temperatures below certain set points. Accordingly, the instrumented blanket 400 may be capable of reducing or eliminating both cold and hot spots in associated processing equipment, using the heating structure 432 and the cooling structure 440 as described herein.

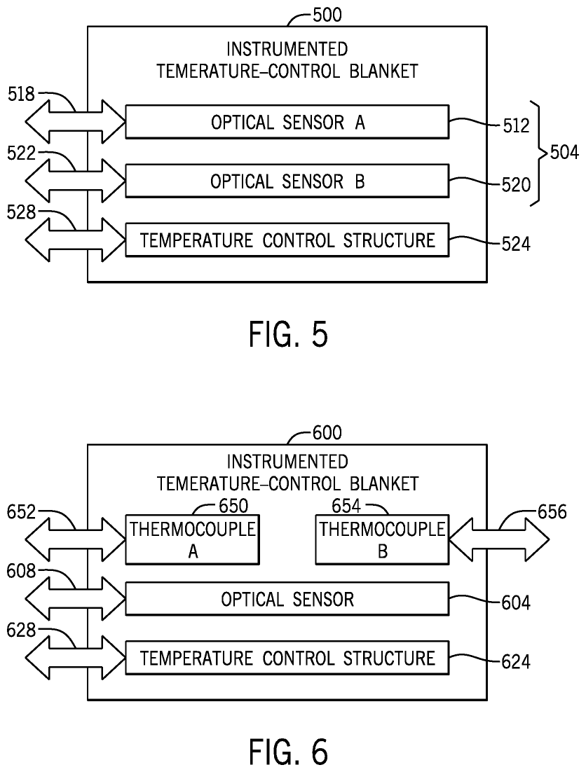

With reference to FIG. 5, an instrumented blanket 500 is shown. The instrumented blanket 500 may be substantially analogous to the instrumented temperature control blankets 300 and 400 of FIGS. 3 and 4, respectively, and include optical sensors 504, a temperature control structure 524, and a signal coupling 528. Notwithstanding the foregoing similarities, the instrumented blanket 500 is shown as including both an “optical sensor A” 512 (and associated signal coupling 518) and an “optical sensor B” 520 (and associated signal coupling 522). The instrumented blanket 500 may therefore be capable of measuring changes in optical properties (and determining temperature changes therewith) using two independent optical sensing devices. Accordingly, the optical sensors 512, 522 may cooperate to provide a higher-resolution heating mapping of processing equipment and/or piping associated with the blanket 500. Further, the optical sensors 512, 522 may provide redundancy such that that the blanket 500 remains capable of being used to determine temperature changes and for generating the 3-D temperature map even where one of the optical sensors 512, 522 fails.

With reference to FIG. 6, an instrumented blanket 600 is shown. The instrumented blanket 600 may be substantially analogous to the instrument blankets 300, 400, 500 of FIGS. 3-5, respectively, and include an optical sensor 604 (and associated signal coupling 608) and a temperature control structure 624 (and associated signal coupling 628). Notwithstanding the foregoing similarities, the instrumented blanket 600 is shown as further include a “thermocouple A” 650 (and associated signal coupling 652) and a “thermocouple B” 654 (and associated signal coupling 656). The thermocouples 650, 654 may be used to determine a point-source measurement of temperature of the blanket 600. For example, the thermocouple 650 may be used to determine a temperature of the blanket 600 at a first location, and the thermocouple 654 may be used to determine a temperature of blanket 600 at a second location. In some cases, the temperature measured by each of the respective thermocouples may be used to calibrate the optical sensor 604 and/or other sensing structure and/or other associated processing equipment. As one example, the optical sensor 604 may detect an optical property associated with returned light scattering from an optical fiber for a given, known temperature of the blanket 600 as measured by one of the thermocouples 650, 656. Said optical property may be used to establish a known or baseline optical property for said measured temperature. In this regard, the optical sensor 604 may subsequently measure a change in the optical properties from the optical fiber, according to the methods described herein. Said change in the optical property, in one example, may be compared to the baseline optical property in order to determine the temperature change in the blanket associated with the measured change in the optical property. The presence of multiple thermocouples (e.g., thermocouples 650, 654, and so on) integrated with the blanket 600 and along a length of the optical fiber may provide additional basis for calibrating one or more of the optical sensors, thereby facilitating the enhancement of the precisions and accuracy of the optical sensors.

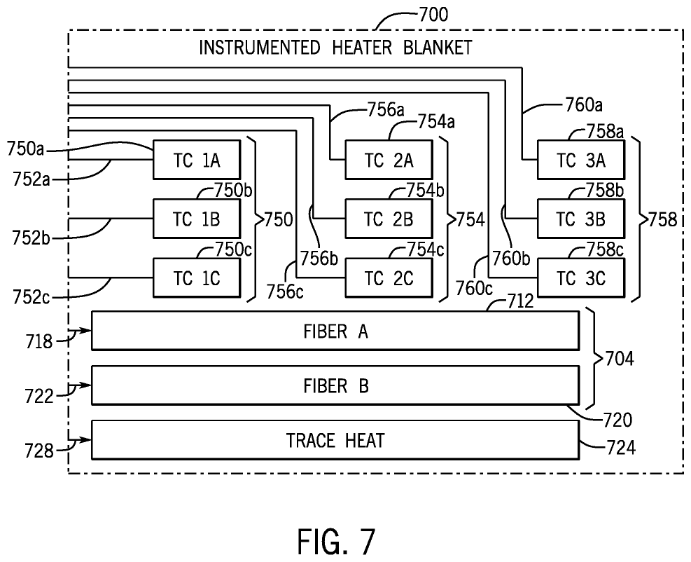

With reference to FIG. 7, an instrumented blanket 700 is shown. The instrumented blanket 700 may be substantially analogous to the instrumented blankets 300, 400, 500, 600 of FIGS. 3-6, respectively, and include optical sensors 704 (including, optical sensor 712 and associated signal coupling 718, and optical sensor 720 and signal coupling 722), and a temperature control structure 724 (including trace heating element and associated signal coupling 728). The blanket 700 may be further analogous to the foregoing blankets in that it includes various thermocouples to detect a temperature point-source temperature of the blanket along a length of the optical fibers 712, 720. Notwithstanding the foregoing similarities, the blanket 700 is shown in FIG. 7 as including multiple thermocouple assemblies, a first thermocouple assembly 750, a second thermocouple assembly 754, and a third thermocouple assembly 758. Each of the thermocouple assemblies 750-758 may include multiple individual thermocouples, such as the thermocouples 650, 654 described above in relation to FIG. 6. For example, the first thermocouple assembly 750 is shown as including thermocouples 750a-750c (and associated signal couplings 752a-752c). Further, the second thermocouple assembly 754 is shown as including thermocouples 754a-754c (and associated signal couplings 756a-756c). Further, the third thermocouple assembly 758 is shown as including thermocouples 758a-758c (and associated signal couplings 760a-760c). Each of the respective thermocouple assemblies 750, 754, 758 may be arranged at a particular point along the length of the optical fibers, such the individual thermocouples of each thermocouple assembly are generally grouped or clustered together with one another (as shown in greater detail with reference to FIG. 10 herein). In this regard, each thermocouple assembly may be used to provide a point-source measurement of the temperature of the blanket 700, which may be used to facilitate the calibration of the optical sensors 704, as described herein. Further, such point-source measurement of temperature for each thermocouple assembly may be provided, collectively, by the associated grouping of individual thermocouples. In this manner, the grouping of thermocouples provides redundancy to the point-source measurement such that any given thermocouple assembly may provide a point-source measurement even where one or two of the individual thermocouples of the assemblies fails. Such redundancy of the thermocouples (and also the optical fibers 712, 720, as shown in FIG. 7) may increase the reliability of the blanket 700, and support its use in harsh environments, such as those associated with molten salt nuclear reactors.

With reference to FIG. 8, a functional diagram of an example system 800 is shown including the instrumented blanket 700 described above in relation to FIG. 7. The blanket 700 is shown coupled with field equipment 802. The field equipment 802 may broadly include any appropriate equipment, including computing equipment, processing units, memory, signal processors, interfaces and the like the facilitate one or more of the functions of the blanket 700, as described herein. While one example of such field equipment is presented for purposes of illustration in FIG. 8, it will be appreciated that in other examples, other and/or different equipment may be used.

In the example of FIG. 8, the field equipment 802 is shown as including processing unit(s) 806, thermocouple or “TC” interface 810, signal generator/receiver 814. and heater power controller 818. Broadly, the processing unit(s) 806 may include one or more computer processors or microcontrollers that are configured to perform operations in response to computer-readable instructions. The processing unit(s) 806 may be a central processing unit of a computer or computing device. Additionally or alternatively, the processing unit(s) 806 may be or be associated with other processors within the device including application specific integrated chips (ASIC) and other microcontroller devices. The processing unit(s) 806 may be coupled to a computer memory and computer readable instructions, which the processing unit(s) 806 may use to perform one or more of the functions described herein.

The TC interface 810 may include a series of electrical connections, signal processors, amplifiers, conditions, and the like. Such components of the TC interface 810 may generally cooperate to receive individual signals from the various thermocouples of the blanket 700 and to produce an output or signal associated therewith for delivery to the processing unit(s) 806. For example, and as shown in FIG. 8, the thermocouple assemblies 750, 754, 758 may generally define a thermocouple array 759. Signals from each thermocouple of the array 759 may be delivered to the TC interface 810 via a wire bundle 761. In some cases, the wire bundle 761 may include some or all of the signal couplings 752a-752c, 754a-754c, 760a-760c described above in relation to FIG. 7. The TC interface 810 may in turn take individual signals from each of said signal couplings and deliver information concerning said signals to the processing unit(s) 806 via operative coupling 811. For example, the TC interface 810 may deliver information to the processing unit(s) 806 concerning a temperature reading from one or more of the thermocouples of the array 759. The processing unit(s) 806 may, in turn, used said information to calibrate the measurements of the optical sensors 704, as described herein.

The field equipment 802 is further shown as including a signal generator/receiver module 814. The module 814 may generally include any appropriate collection of components configured to send and receive signal with the optical fibers 712, 720 of the optical sensors 704, for example, via signal couplings 718, 722. For example, the module 814 may include a signal generator that is configured to propagate an optical signal through one or both of the fibers 712, 720. The module 814 may further include a signal receiver that is configured to detect returned light scattering, based on said optical signals, from the optical fibers 712, 720. For example, the signal receiver may be configured to detect said light scattering from each interval of a plurality of intervals of the optical fibers 712, 720. In this regard, and as described herein, the signal generator and receiver may cooperate to detect changes in optical properties associated with each of the fibers 712, 720 by sending and receiving such signals through the fibers. The module 814 may in turn take information associated with the returned light scattering and deliver said information to the processing unit(s) 806 via operative coupling 815. Upon receipt of such information at the processing unit(s) 806, the processing unit(s) 806 may analyze the returned light scattering to determine the change in the optical property from each interval of the plurality of intervals along a length of each respective optical fiber 712, 720. Further, the processing unit(s) 806 may determine the temperature of the blanket 700 for each interval of the plurality of intervals by comparing the change in the optical property for each respective interval with a baseline optical property (e.g., a baseline optical property derived using the thermocouple array 759). Furthermore, in some cases, the processing unit may additionally or alternatively determine a strain, stress, and/or neutron or gamma fluence associated with said location by comparing of the foregoing parameters with a baseline optical property (e.g., a known light frequency spectrum for a given strain, stress, or neutron or gamma fluence).

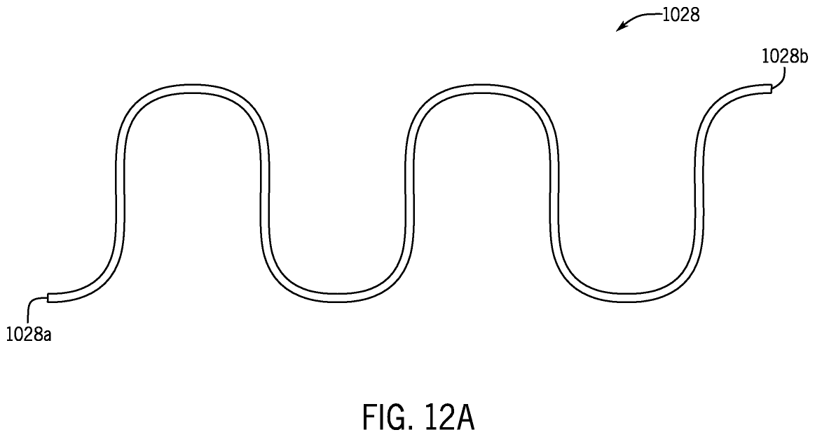

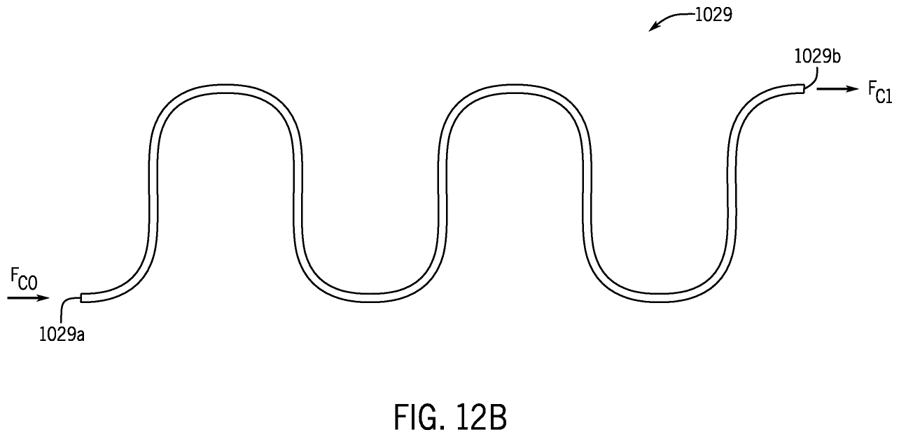

The field equipment 802 is further shown as including a heat power controller module 818. The heat power control module 818 may include any appropriate collection of components, assemblies, and subsystem used to provide an input for controlling the temperature control structure 724. For example, the module 818 may include an amplifier, controller, and other signal processing components that are adapted to deliver an input electrical signal to the trace heating element of the temperature control structure 724 via the signal coupling 728. In this regard, the module 818 may vary the input electrical signal in order to vary an intensity (e.g., a heat output) of the trace heating element. The input electric signal may be delivered to the trace heating element in response to a signal from the processing unit(s) 806. For example, the module 818 may receive one or more signals from the processing unit(s) 806 via operative coupling 819 that cause the module 818: (i) to send an input electrical signal to the trace heating element that increases a heating output of the temperature control structure 724, and/or (ii) to send an input electrical signal to the trace heating element that decreases a heating output of the temperature control structure 724. As described herein, the input electrical signal may be calibrated to increase a heat output of the trace heating element where a temperature of the blanket 700 is measured as being below one or more set points, and the input electrical signal may be calibrated to decrease a heat output of the trace heating element where a temperature of blanket 700 is measured as being above one or more set points. Further, it will be appreciated that while a trace heating element is shown for the temperature control structure 724 for purposes of illustration, the temperature control structure may also include certain cooling elements or mediums (e.g., such as the cooling structure shown in relation to FIG. 12B), which may be controlled in an analogous manner by the module 818.

The system 800 is further shown with the field equipment 802 coupled to control room computing device 840 via an operatively coupling 807. The control room computing device 840 may be configured to perform one or more of the functions of the filed equipment 802 described herein. In this regarding the control room computing device 840 may include processing unit(s) 844. The processing unit(s) 844 may be substantially analogous to the processing unit(s) 806; redundant explanation of which is omitted herein for clarity. In some cases, the control room computing device 840 may receive data from the field equipment 802 for purposes of generating one or more of the 3-D temperature map described herein. For example, the process unit(s) 844 and/or other computing devices may be configured to generate a 3-D temperature map corresponding to a 3-D surface of a pipe, vessel, and/or other processes component with which the blanket 700 is associated. In some cases, the information from the 3-D temperature map may be shared with other components or system of the process in order to perform or change one or more operations. For sake of non-limiting example, the information from the 3-D temperature map may be shared with a pump, mixer, a control valve and/or other component which may be responsive to temperature changes within the systems within which the blanket 700 is associated.

With reference to FIG. 9, a functional diagram 900 of certain P&ID logic of the system 800 of FIG. 8 is shown. For example, the system 800 may operate to continually adjust a temperature of the blanket 700 based on temperature reading from the optical sensors integrated therewith. In one example, the field equipment 802 may execute the P&ID logic of diagram 900 using one or more or all of the processing unit(s) 806, the TC interface 810, the signal generator/receiver module 814, and/or the heat power controller module 818, described herein above. In operation, as shown in FIG. 9, a temperature value may be set at “set value” function 904, such as setting the temperature value to an ideal process temperature for a molten salt at 650° C. Subsequently, the temperature value may be compared to a measured temperature value (delivered by the “measure” function 924) at the “comparator” function 908. The measured temperature valve may a temperature value as determined using the optical sensors 704, according to the method described herein. Where the set temperature deviates from the measured temperature, the operation shown in FIG. 9 proceeds to the “controller” operation 912, in which the heat power controller module 818 may generate an input electrical signal based on whether the heat trace should proceed more or less heat. Said input electrical signal is received by a “control element” function 916 (e.g., the temperature control structure 724) to cause said heat changes in the blanket 700 using the electrical signal. Subsequently, the function of FIG. 9 may proceed to the “process” function 920 at which the pipe, vessel and/or processing unit continues operation, as influenced by the heat output of the temperature control structure 724. Subsequently, the function of FIG. 9 may proceed to “measure” function 924 where at the optical sensors 704 may continue to measure the temperature of the blanket 700 according to the method described herein. In this manner, system 800 may continually change one or more heat output parameters of the blanket 700 based on the ongoing changes with an associated process or system. Accordingly, the blanket 700 may be used to identify in near real-time cold- or hot-spots in an associated process or system, and to mitigate such cold- or hot-spots by altering the heat output of the blanket 700 correspondingly.

With reference to FIG. 10, an instrumented blanket 1000 is shown. The instrumented blanket 1000 may be substantially analogous to any of the temperature control blankets described herein, and may include an insulative material 1002, thermocouple assemblies 1006a, 1006b, 1006c, a temperature control structure including a heating structure 1028 and a cooling structure 1032, and optical fibers 1036a, 1036b. FIG. 10 shows the blanket 1000 with the heating structure 1028, cooling structure and optical fibers 1036a, 1036b arranged along a serpentine path 1020; although, other paths and arrangements are possible. With further reference to FIG. 10, the serpentine path 1020 is shown progressing along a longitudinal axis 1010 of the blanket 1000. Each of the thermocouple assemblies 1006a, 1006b, 1006c may be arranged at a respective position relative to the longitudinal axis 1010 in order to facilitate the calibration operations of the optical fibers 1036a, 1036b, described herein. For example, the first thermocouple assembly 1006a is shown arranged at a first longitudinal location 1014a, the second thermocouple assembly 1006b is shown arranged at a second longitudinal location 1014b, and the third thermocouple assembly 1006c is shown arranged at a third longitudinal location 1014c. In this regard, the blanket 1000 may provide a point-source temperature measurement at each of the longitudinal locations 1006a-1006c. Each of the longitudinal locations 1006a-1006c may be associated with a certain interval of the plurality of interval of each optical fiber 1036a, 1036b such that a processing unit may be used to measure an optical property of said optical fibers 1036a, 1036b for a known temperature, as known per the corresponding thermocouple assembly arranged at said longitudinal location, to establish a baseline optical property for said temperature. Accordingly, upon a change in the temperature of the blanket 1000, which may induce a change in optical properties as measured through the respective optical fibers 1036a, 1036b, said change in optical properties can be compared relative to the baseline optical property in order to determine the change in temperature of the blanket 1000.

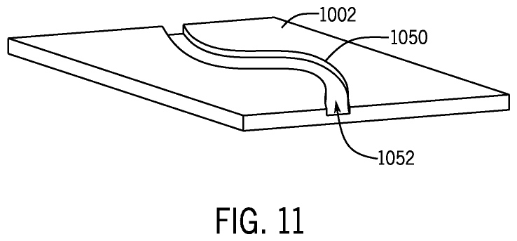

Turning to FIGS. 11-15, select example components of the blanket 1000 are shown and described for purposes of illustration. In other cases, other components may be used to facilitate one or more of functions described herein. With reference to FIG. 11, the insulative material 1002 is shown. The insulative material 1002 may be a ceramic, wool and/or other material configured to withstand sufficiently high temperatures and to block heat from escaping therethrough. The insulative material 1002 may be single or multilayer material. In some cases, the insulative material 1002 may be arranged in a jacket or outer sleeve. At least some portion of the insulative material 1050 may be configured to receive the various optical sensors and temperature control structures described herein. For example, and as shown in FIG. 11, the insulative material 1002 may include formed channels 1050 therein that define a channel volume 1052. The formed channels 1050 may be configured to receive at least a portion of an optical fiber of the optical sensor, at least a portion of a heat trace, and/or at least a portion of another element of the temperature control structure, including elements of a cooling structure. The formed channels 105 may generally be arranged in a serpentine pattern, as shown in FIG. 11; although in other cases, other arrangements are possible. Further, in some cases, each of the optical fibers and temperature control structure may be arranged in a single formed channel, whereas in other cases multiple such channels may be used that area configured specific ones of the optical fibers and/or temperature control structures.

With reference to FIG. 12A, an example heat trace 1028 is shown of the instrumented blanket 1000 of FIG. 10. The heat trace 1028 may be a nickel/nichrome wire or heating resistance ribbon having a first end 1028a and a second end 1028b. For example, an 80 nickel-20 chromium wire may be used that is capable of producing above 700° C. in response to an input electrical signal. The heat trace 1028 may be such that that the intensity or other properties of the input electrical signal may be varied in order to vary the heat output of the heat trace 1028.

With reference to FIG. 12B, an example cooling structure 1029 is shown for use in the instrumented blanket 1000 of FIG. 10. For example, the cooling structure 1029 may generally include a flexible bladder or flexible conduit capable of routing a flow of a cooling medium therethrough in response to a receipt of a cooled flow of said medium into the flexible material. In this regard, the cooling structure 1029 is shown in FIG. 12B as having a first end 1029a capable of receiving a flow F_{c0} of cooling medium into the flexible bladder. The cooling structure 1029 is further shown in FIG. 12B as having a second end 1029b capable of emitting an exit flow F_{c1} from the flexible bladder.

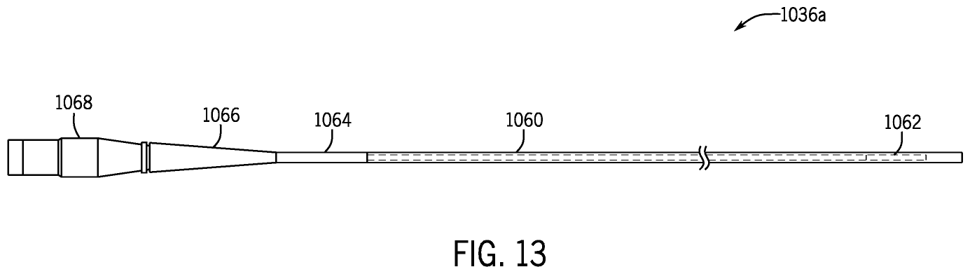

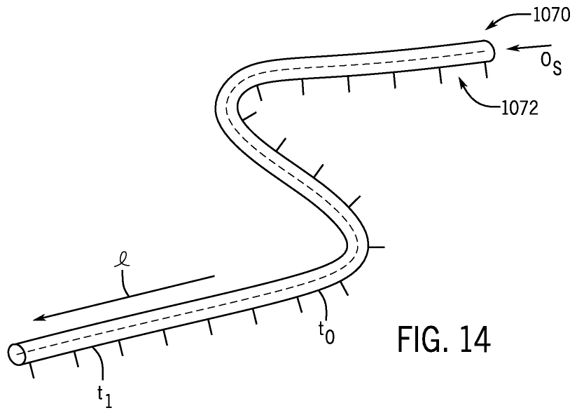

With reference to FIG. 13, an example optical sensor 1036a is shown. The optical sensor 1036a may include a connector section 1068, a transition piece 1066, a fiber mount 1064, an optical fiber section 1060, and a termination tip 1062. The optical sensor 1036a, as described herein, may be configured to propagate optical signals along an optical fiber of the optical fiber section 1060, and detect returned light scattering from said light signals. In this regard, the connector 1068 may include optical receptors, pins, and/or other types of connectors that can be used to couple the optical sensor 1036a to the signal generator/receiver module 814. The transition piece 1066 may operate to transfer such signals to and from the signal generator/receiver module 814 and to the optical fiber. The fiber mount 1064 may provide a mount from which the optical fiber 1070 of the optical fiber section 1060 sections. The termination tip 1062 may represent an endpoint of the optical fiber 1070 within the optical fiber section 1060. With reference to FIG. 14, an example optical fiber 1070 is shown. The optical fiber 1070 may be integrated within the optical fiber section 1060. The optical fiber 1070 may include a plurality of interval 1072 along the length 1 of the fiber 1070. The optical fiber 1070 may be configured to receive an optical signal Oₛ at an end of the fiber 1070 (e.g., as may be generated or prompted by the signal generator/receiver module 814). The optical signal Oₛ may travel along a length of the optical fiber 1070, as indicated by the dashed line shown in FIG. 14. As the optical signal Oₛ travels along a length of the optical fiber 1070, at least a portion of the signal may scatter back along the fiber 1070, toward the origin of the optical signal Oₛ. The optical sensors, as described herein, are configured to detect and measure said returned light scattering relative to each interval of the plurality of intervals 1072. Further, the optical sensors, as described herein, are configured to detect a time of the detected returned light scattering based at least in part on the propagation time of the Oₛ through the optical fiber 1070 (e.g., where a propagation time of the Oₛ to a first interval is t₀, whereas a propagation time of the Oₛ to a second interval if t₁, and so on).

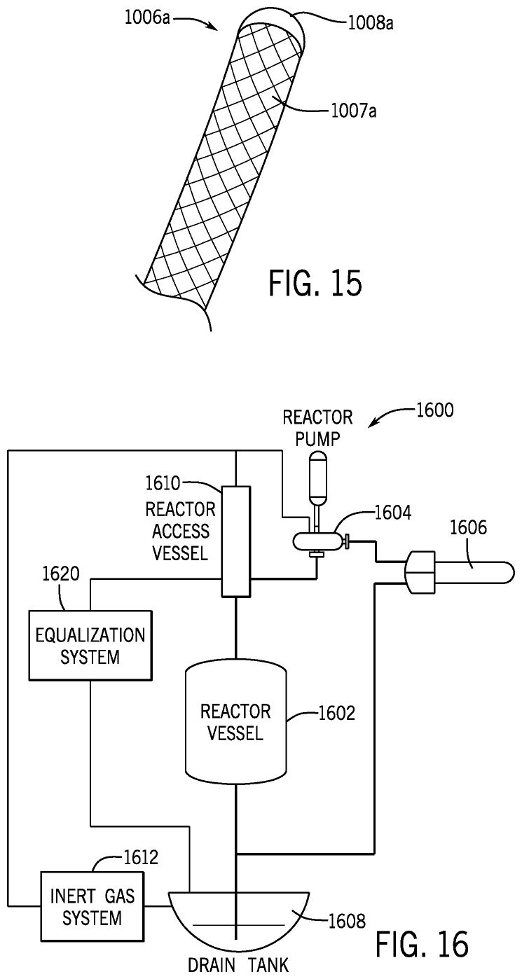

With reference to FIG. 15, an example thermocouple 1006a is shown. The thermocouple 1006a may include a braiding 1007a and a probe end 1008a. The probe end 1008a may be configured to measure temperature of over 1000° C. The braiding 1007a may provide a surface insulation for the internal wire leads of the thermocouple 1006a. In other cases, other types of thermocouple may be used, includes those with through holes and/or other features that facilitate clamping or otherwise securing of the thermocouple within any of the blankets described herein.

FIG. 16 depicts a schematic representation of an example molten salt reactor system 1600. The molten salt reactor system 1600 may implement and include any of the instrumented blankets described herein, and implement any of the functionalities thereof. As will be understood, the example shown in FIG. 16 represents merely one example configuration of a molten salt reactor system 1600 in which such inert gas systems and equalization systems may be utilized. It will be understood that the inert gas systems and the equalization systems described herein may be used in and with substantially any other configuration of the molten salt reactor, as contemplated herein.

In various embodiments, a molten salt reactor system 1600 utilizes fuel salt enriched with uranium (e.g., high-assay low-enriched uranium) to create thermal power via nuclear fission reactions. In at least one embodiment, the composition of the fuel salt may be LiF—BeF₂—UF₄, though other compositions of fuel salts may be utilized as fuel salts within the reactor system 1600. The fuel salt within the system 1600 is heated to high temperatures (about 600° C. or higher) and melts as the system 1600 is heated. In several embodiments, the molten salt reactor system 1600 includes a reactor vessel 1602 where the nuclear reactions occur within the molten fuel salt, a fuel salt pump 1604 that pumps the molten fuel salt to a heat exchanger 1606, such that the molten fuel salt re-enters the reactor vessel after flowing through the heat exchanger, and piping in between each component. The molten salt reactor system 1600 may also include additional components, such as, but not limited to, drain tank 1608 and reactor access vessel 1610. The drain tank 1608 may be configured to store the fuel salt once the fuel salt is in the reactor system 1600 but in a subcritical state, and also acts as storage for the fuel salt if power is lost in the system 1600. The reactor access vessel may be configured to allow for introduction of small pellets of uranium fluoride (UF₄) to the system 1600 as necessary to bring the reactor to a critical state and compensate for depletion of fissile material.

In several examples, the molten salt reactor system 1600 may include an inert gas system 1612 to provide inert gas to a head space of the drain tank 1608, among other functions. The inert gas system 1612 may further relieve inert gas from the head space of the drain tank 1608 as needed. The inert gas system 1612 is therefore operable to maintain pressurized inert gas in the head space of the drain tank 1608 that is sufficient to substantially prevent the flow of molten fuel salt into the drain tank during normal operations. For example, with the head space of the drain tank 1608 pressurized by the inert gas system 1612, molten salt may generally circulate between the reactor vessel 1602 and the heat exchanger 1606 without substantially draining into the drain tank 1608. As described herein, the inert gas system 1612 may be configured to supply inert gas to the head space of various other components of the molten salt reactor system 1600, such as to the head space of the reactor access vessel 1610, to the seal of reactor pump 1604, among other components. Upon the occurrence of a shutdown event, the inert gas system 1612 may cease providing inert gas to the head space of the drain tank 1608, and other components to which the system 1612 supplies inert gas.

The molten salt reactor system 1600 may further include an equalization system 1620 that is operable to equalize the pressure among all headspace of the system 1600, including, without limitation, the head space of the drain tank 1608 and the reactor vessel 1602 upon the occurrence of a shutdown event. For example, during normal operation, a pressure differential exists between the head space of the drain tank 1608 and the reactor vessel 1602. Such pressure differential prevents or impedes the draining of the fuel salt into the drain tank 1608. In this regard, the equalization system 1620 may be operable to fluidically couple (via opening one or more valves) the head space of the drain tank 1608 and the reactor vessel 1602 to reduce or eliminate the pressure differential, thereby allowing the fuel salt to readily flow into the drain tank upon the shutdown event. The equalization system 1620 may include numerous redundancies and/or bypasses in order to facilitate a fail-safe or walk-away safe operation with respect to depressurization of the system 1600.

The instrumented blankets of the present disclosure may be used with any pipe, vessel, and/or process equipment of the molten salt reactor system shown with reference to FIG. 16. In one example, such instrumented blanket may be used to insulate and control the temperature of a pipe of the primary fuel salt loop. For example, an instrumented blanket may be used on a pipe between the reactor vessel 1602 and the reactor access vessel 1610, a pipe between the reactor access vessel 1610 and the reactor pump 1604, a pipe between the reactor pump 1604 and the heat exchanger 1606, a pipe between the heat exchanger 1606 and the reactor vessel 1602/drain tank 1608, and a pipe between the drain tank 1608 and the reactor vessel 1602. The molten salt along such fuel loop remains in molten form during operation of the system 1600. The blankets described herein may be used to determine, in real-time a temperate of such pipes and to generate a 3-D temperature mapping of said pipes (e.g., such as the temperature map shown in FIG. 2, herein). Additionally or alternatively, the blankets described herein may be used to determine a strain, stress, and/or neutron or gamma fluence associated with such pipes and to generate 3-D of said parameters. In operation, such 3-D temperature maps of the pipes of the fuel loop may be used to determine a cold- or hot-spots of the molten salt, and to mitigate such cold- or hotspots by increasing or decreasing a heat output of the blanket, as appropriate. In this regard, the instrumented blankets of the present disclosure may be used to ensure the molten salt maintains a temperature consistency that supports efficiency reactor operations.

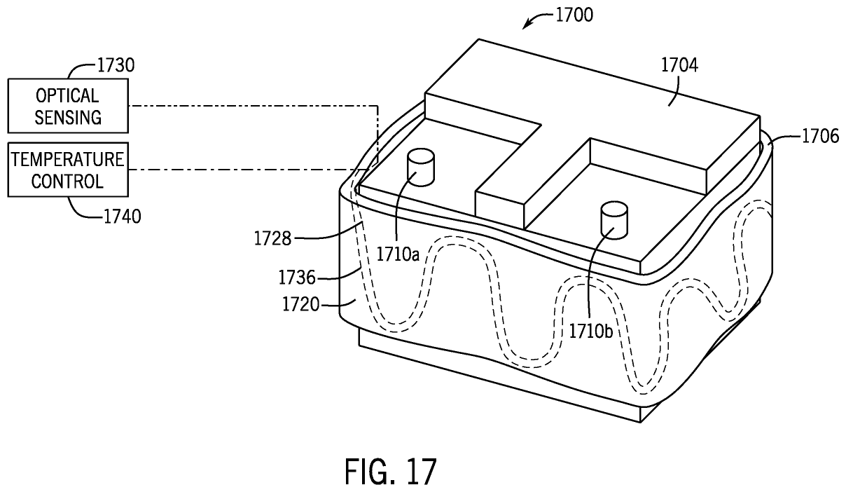

While the instrumented blankets of the present disclosure are described above with reference to nuclear reactors, it will be appreciated that such blankets may be used with generally any type of component in which it is desirable to measure and maintain a temperature of said components. As one non-limiting example, FIG. 17 depicts a system 1700 including a battery 1704 and an instrumented blanket 1720. The battery 1704, for purposes of illustration, may be an automotive battery having a battery surface area 1706 and terminals 1710a, 1710b. In order to promote the efficient operation of the battery 1704, it may be desirable to monitor a temperature distribution of the battery 1704 across the battery surface area 1706, and to provide temperature control (e.g., cooling) to the battery 1704, if needed.

In this regard, the provided instrumented blanket 1720 may be substantially analogous to any of the instrumented blankets described herein and may include a temperature control structure 1736 (and associated temperature control module 1740) and an optical sensor 1728 (and associated optical sensing module 1730) arranged within an insulative material of the blanket 1720; redundant explanation of which is omitted here for clarity. In operation, the blanket 1720 may use the optical sensor 1728 to detect changes in optical properties from one or more optical fibers integrated therewith that are indicative of temperature changes in the blanket 1720 (and in the battery 1704). In turn, where the temperature changes exceed or otherwise deviate from one or more set points, the blanket 1720 may use the temperature control structure 1736 to alter a temperature output of blanket 1720 to move the measured temperature toward the one or more set points accordingly. For example, where the blanket 1720 indicates a battery overheating condition and/or other hots-spots along the surface 1706, the blanket 1720 may cause one or more cooling structure of the temperature control structure 1736 to producing a cooling output to cool the battery 1704.

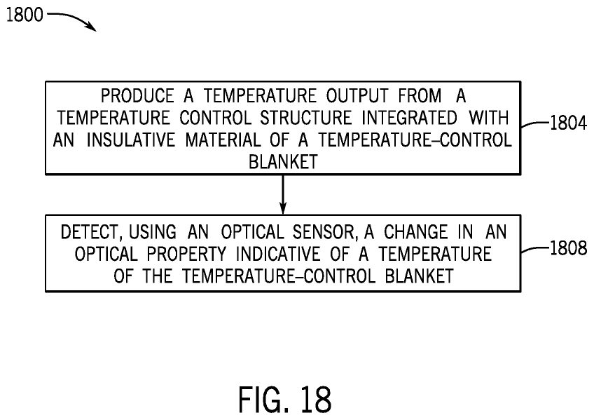

FIG. 18 depicts a flow diagram of an example method 1800 of operating an instrumented blanket is depicted. At operation 1804, a temperature output is produced from a temperature control structure that is integrated with an insulative material of a blanket. For example, and with reference to FIG. 1, the heating structure 136 is integrated with the insulative material 120 of the instrumented blanket 120. The heating structure 126 is configured to produce a heat output in response to an input electric signal generated by the temperature control module 140. At operation 1808, a change in an optical property is detected, using an optical sensor, that is indicative of a temperature of the blanket. For example, and with continued reference to FIG. 1, a change in an optical property is detected from the optical fiber 128. The change in the optical property may be indicative of a change in a temperature of the blanket 120. In this regard, the optical sensing module 132 may determine a change in the temperature of the blanket 120 using said optical sensor, as described herein. Further, and as described herein, the optical sensing module 132 may cause, based on the determined temperature deviating from one or more set points, the temperature control module 140 to alter a characteristic of the input electric signal in order to increase or reduce the heat output from the heating structured 126, as needed.

Other examples and implementations are within the scope and spirit of the disclosure and appended claims. For example, features implementing functions may also be physically located at various positions, including being distributed such that portions of functions are implemented at different physical locations. The foregoing description, for purposes of explanation, uses specific nomenclature to provide a thorough understanding of the described examples. However, it will be apparent to one skilled in the art that the specific details are not required in order to practice the described examples. Thus, the foregoing descriptions of the specific examples described herein are presented for purposes of illustration and description. They are not targeted to be exhaustive or to limit the examples to the precise forms disclosed. It will be apparent to one of ordinary skill in the art that many modifications and variations are possible in view of the above teachings.

What is claimed is:

1. An instrumented temperature-control blanket comprising
an insulative material;
a temperature control structure integrated with the insulative material and configured to produce a heat or a cooling output in response to an input signal; and
an optical sensor including an optical fiber integrated with the insulative material along the heating structure,
wherein the optical sensor is configured to detect a change in an optical property measured from a plurality of intervals along a length of the optical fiber, the change in the optical property indicative of a temperature of the temperature-control blanket at a respective interval of the plurality of intervals.

2. The blanket of claim 1, wherein
the optical sensor comprises
a signal generator configured to propagate an optical signal through the optical fiber, and
a receiver configured to detect returned light scattering, based on the optical signal, from each interval of the plurality of intervals of the optical fiber, and
the optical sensor is coupled with a processing unit configured to
analyze the returned light scattering to determine the change in the optical property from each interval of the plurality of intervals along the length of the optical fiber, and
determine the temperature of the temperature-control blanket for each interval of the plurality of intervals by comparing the change in the optical property for each respective interval with a baseline optical property.

3. The blanket of claim 2, wherein the processing unit may be configured to analyze the returned light scattering using one or both of an optical time domain reflectometry or an optical frequency domain reflectometry.

4. The blanket of claim 2, wherein
the optical property comprises a light frequency spectrum, and
the processing unit associates changes of the light frequency spectrum with each respective interval of the plurality of intervals based on a detected time of the returned light scattering at the receiver.

5. The blanket of claim 2, wherein
the insulative material is wrapped around a pipe or a vessel, and
the processing unit is configured to generate a 3-D temperature map corresponding to a 3-D surface of the pipe or vessel engaged with the insulative material, the 3-D temperature map formed from the determined temperature of the temperature-control blanket at each respective interval.

6. The blanket of claim 2, wherein
the processing unit is configured to compare the determined temperature of the temperature-control blanket to one or more set points,
the blanket further comprises a controller operatively coupled with the temperature control structure and configured to deliver the input signal thereto, and
the controller is configured to change a property of the input signal based on the determined temperature of the temperature-control blanket deviating from the one or more set points, the input signal comprises at least one of an electrical signal or a flow of a coolant medium.

7. The blanket of claim 2, wherein
the temperature control structure comprises a heating structure and the blanket further comprises
a first thermocouple assembly integrated with the insulative material along a first interval of the plurality of intervals of the optical fiber and configured to measure a first temperature of the temperature-control blanket at the first interval, and
a second thermocouple assembly integrated with the insulative material along a interval of the plurality of intervals of the optical fiber and configured to measure a second temperature of the temperature-control blanket at the second interval, and
the processing unit is configured to determine the baseline optical property by correlating the first measured temperature of the first thermocouple assembly and the second measured temperature of the second thermocouple assembly with the optical property measured by the optical sensor for said first and second temperatures.

8. The blanket of claim 1, wherein
the optical sensor is a first optical sensor, and
the blanket further comprises
a second optical sensor including a second optical fiber integrated with the insulative material along the temperature control structure and the first optical sensor,
wherein the second optical sensor is configured to detect a change in an optical property measured from a plurality of intervals along a length of the second optical fiber, the change in the optical property indicative of a temperature of the temperature-control blanket at a respective interval of the plurality of intervals of the second optical fiber.

9. The blanket of claim 1, wherein the insulative material comprises a high-temperature ceramic with formed channels therein configured to receive at least a portion of the temperature control structure and at least a portion of the optical fiber.

10. The blanket of claim 9, further comprising high-temperature ceramic threads engaged with the insulative material and the portion of the temperature control structure and/or the portion of the optical fiber to secure the portion of the temperature control structure and/or the portion of the optical fiber in the formed channels.

11. The blanket of claim 9, wherein the portion of the temperature control structure and the portion of the optical fiber are enclosed within the insulative material.

12. The blanket of claim 11, wherein the formed channels define a serpentine pattern.

13. The blanket of claim 2, wherein the temperature control structure comprises a heating structure and a cooling structure integrated with the insulative material.

14. The blanket of claim 13, wherein
the processing unit is configured to compare the determined temperature of the temperature-control blanket to one or more set points,
the blanket further comprises a controller operatively coupled with the temperature control structure configured to deliver the input signal to the heating structure and/or the cooling structure, and
the controller is configured to change a property of the input signal based on the determined temperature of the temperature-control blanket deviating from the one or more set points.

15. A system comprising
the blanket of claim 1; and
a pipe or a vessel having a fluid therein,
wherein the blanket is engaged with the pipe or the vessel to provide the heat output of the heating structure to the fluid.

16. The system of claim 15, wherein the blanket further comprises
a signal generator configured to propagate an optical signal through the optical fiber, and
a receiver configured to detect returned light scattering, based on the optical signal, from each interval of the plurality of intervals of the optical fiber, and
the system further comprises
a processing unit configured to
analyze the returned light scattering to determine the change in the optical property from each interval of the plurality of intervals along the length of the optical fiber, and
determine the temperature of the temperature-control blanket for each interval of the plurality of intervals by comparing the change in the optical property for each respective interval with a baseline optical property.

17. The system of claim 16, wherein
the fluid comprises a fissile molten salt fluid of a molten salt reactor system,
the processing unit is further configured to compare the determined temperature of the temperature control blanket to one or more set points associated with a freezing temperature of the fissile molten salt fluid,
the blanket further comprises a controller operatively coupled with the heating structure and configured to deliver the input electrical signal thereto, and
the controller is configured to change a property of the input electrical signal based on the determined temperature of the temperature-control blanket advancing toward the one or more set points.

18. A method of operating an instrumented temperature-control blanket, the method comprising
producing a heat output from a temperature control structure in response to an input signal, the heating structure integrated with an insulative material; and
detecting, using an optical sensor including an optical fiber integrated with the insulative material along the temperature control structure, a change in an optical property measured from a plurality of intervals along a length of the optical fiber, the change in the optical property indicative of a temperature of the temperature-control blanket at a respective interval of the plurality of intervals.

19. The method of claim 18, further comprising
propagating, using a signal generator of the optical sensor, an optical signal through the optical fiber,
detecting, using a receiver of the optical sensor, a returned light scattering, based on the optical signal, from each interval of the plurality of intervals of the optical fiber,
analyzing, using a processing unit coupled with the optical sensor, the returned light scattering to determine the change in the optical property from each interval of the plurality of intervals along the length of the optical fiber, and
determining, using the processing unit, the temperature of the temperature-control blanket for each interval of the plurality of intervals by comparing the change in the optical property for each respective interval with a baseline optical property.

20. The method of claim 19, further comprising
comparing, using the processing unit, the determined temperature of the temperature control-blanket to one or more set points,
changing, using a controller operatively coupled to the temperature control structure and the processing unit, a property of input signal based on the determined temperature of the temperature-control blanket deviating from the one or more set points.
